(19) United States
(12) Patent Application Publication
(10) Pub. No.: US 2007/0074300 A1
(43) Pub. Date: Mar. 29, 2007

(54) Sialidase inhibitors for the treatment of cardiovascular disease

(76) Inventors: Suleiman Igdoura, Dundas (CA); Bernardo Trigatti, Hamilton (CA)

Correspondence Address: GOWLING, LAFLEUR HENDERSON LLP, ONE MAIN STREET WEST, HAMILTON, ON, L8P 4Z5, CA

(21) Appl. No.: 11/528,333

(22) Filed: Sep. 28, 2006

Related U.S. Application Data
(60) Provisional application No. 60/721,126, filed on Sep. 28, 2005.

(51) Int. Cl. A01K 67/027; A61K 39/395; A61K 31/739; A61K 48/00; A61K 31/56; A61K 31/397; A61K 31/401; A61K 31/366; A61K 31/22

(52) U.S. Cl. 800/18; 514/54; 424/146.1; 514/44; 514/58; 514/423; 514/460; 514/548; 514/171; 514/210.02; 514/356

(57) ABSTRACT

A method of treating or preventing a disorder associated with metabolic syndrome is provided. In particular, methods of treating atherosclerosis and diabetes by reducing sialidase activity are provided.

### FIELD OF INVENTION

The present invention relates to the field of enzymology and diseases related to enzyme activity. More particularly, the invention relates to sialidase inhibitors and their use as agents for the treatment of disorders associated with metabolic syndrome, such as high LDL cholesterol, cardiovascular disease and diabetes.

### BACKGROUND OF THE INVENTION

Atherosclerosis is a major cause of cardiovascular disease and stroke in modern society. Atherosclerosis often occurs in the context of other diseases, including diabetes, obesity, and hypertension. This constellation of diseases is referred to as the Metabolic Syndrome. This is sometimes referred to as Insulin Resistance Syndrome (IRS). IRS usually involves the concomittant existence in a subject of two or more of dyslipidemia, hypertension, type 2 diabetes, impaired glucose tolerance, hyperuricaemia, a pro-coagulant state atherosclerosis and truncal obesity.

A number of factors contribute to atherosclerosis susceptibility. Among the most important of these are the levels of cholesterol associated with different classes of lipoproteins circulating in blood. Epidemiological evidence has demonstrated that “HDL-C”, the level of cholesterol associated with high density lipoproteins (HDL) in blood plasma or serum is negatively correlated with risk, whereas “LDL-C”, the level of cholesterol associated with low density lipoproteins (LDL) in blood plasma or serum are directly correlated with risk for atherosclerosis and heart disease.

The sialidases comprise a family of hydrolytic enzymes that cleave sialic acid, an acidic sugar, from glycoproteins, glycolipids and oligosaccharides. Sialic acid is the most abundant terminal monosaccharide on the surface of eukaryotic cells. Due to its strong negative charge, widespread distribution, and predominant terminal position, sialic acid is involved in a variety of important biological activities including cellular differentiation, tumorigenicity and antigen masking.

Lysosomal and cell surface sialidase has been shown to directly de-sialylate surface molecules such as CD44. Katoh et al. (1999) and Gee et al. (2003) have shown that lysosomal sialidase activation is required for the acquisition of the hyaluronic acid (HA)-binding form of CD44 in LPS- and TNFα-stimulated monocytic cells. Sialylation of CD44 N-glycans (Asn25 and Asn120) may either directly block HA binding or reduce receptor avidity by preventing homo-oligomerization (Teriete et al, 2004). LPS-induced sialidase activity appears to be dependent on CD44-HA-binding (Gee et al 2003, Katoh et a 1999). Blocking desialysis of CD44, thus interfering with its ability to bind HA may affect inflammation, atherosclerosis and related conditions.

There is a long-standing recognized need for novel approaches to the treatment of metabolic disease disorders. Although many of the molecules that have been implicated in playing a role in the manifestation of metabolic syndrome related disorders are sialylated, there has been no previous disclosure of therapeutic agents that target this aspect, including control of LDL cholesterol levels, blood glucose levels or development of atherosclerosis. The present invention addresses the need for novel therapeutic approaches for the prevention and/or treatment of metabolic syndrome disorders.

### SUMMARY OF THE INVENTION

The present invention provides a novel therapeutic for the treatment of diseases such as cardiovascular disease and diabetes that are associated with metabolic syndrome. Metabolic syndrome is a term used to refer to a group of metabolic risk factors in an individual. These include altered cholesterol metabolism, insulin resistance or glucose intolerance, abdominal obesity, elevated blood pressure, a prothrombotic state and a proinflammatory state. Atherogenic lipidemia is an important aspect of this syndrome. High triglycerides, low HDL cholesterol and high LDL cholesterol foster atherosclerotic plaque build-up in artery walls and atherosclerosis can lead to coronary heart disease and stroke. The methods, uses and compositions of the present invention are also useful to treat high triglycerides and LDL cholesterol.

The present invention provides a novel method of preventing and/or treating atherosclerosis and other metabolic syndrome disorders by inhibiting endogenous sialidase activity.

In one aspect of the invention, methods for treating or preventing a disorder associated with metabolic syndrome by administering a sialidase inhibitor are provided. In a preferred embodiment, the disorder is selected from the group consisting of atherosclerosis, diabetes, dyslipedemia, glucose intolerance, obesity and hypertension. In a more preferred embodiment, a method of lowering LDL-cholesterol and treating or preventing atherosclerosis is provided. The methods of the present invention may also be used to lower serum levels of glucose.

The methods of the invention are also beneficial for combating other inflammatory diseases. These may include, but are not limited to, various forms of arthritis, asthma, inflammatory bowel disease, Crohn's disease and colitis, inflammatory skin and eye diseases, end stage renal disease, autoimmune disease related systemic inflammation, inflammatory cardiomyopathies, calcified aortic stenosis, chronic obstructive pulmonary disease and others.

In one aspect of the invention, the sialidase inhibitor is administered as a prophylactic measure against cardiovascular disease and associated conditions such as high triglycerides and LDL cholesterol.

In another aspect of the invention, the sialidase inhibitor is administered as a therapeutic measure for cardiovascular disease. The sialidase inhibitor may be administered alone or sequentially or simultaneously with another drug.

In one particularly preferred embodiment, a method of reducing plasma, serum or blood LDL cholesterol levels comprising administering a sialidase inhibitor is provided. The sialidase inhibitor may be administered alone or sequentially or simultaneously with another drug for the treatment of high LDL cholesterol.

In the present invention, the sialidase inhibitor may be administered via any suitable route such as, but not limited to, oral, mucosal, transdermal, subcutaneous, intravenous, intraperitoneal and intramuscular routes. In one preferred embodiment, the sialidase inhibitor is administered orally. In another preferred embodiment, the sialidase inhibitor is provided in a skin patch. In certain situations, the sialidase inhibitor is preferably administered by injection.

In another preferred embodiment, a composition comprising a sialidase inhibitor and a pharmaceutically acceptable excipient is administered to a patient suffering from cardiovascular disease or related conditions such as high LDL cholesterol or triglycerides. In another preferred embodiment, a therapeutic amount of the sialidase inhibitor is administered in combination with another cardiovascular disease treatment agent or a lipid-lowering agent.

In another aspect of the invention, a pharmaceutical composition useful for the treatment of metabolic syndrome—related disorders are provided. The pharmaceutical composition comprises an inhibitor that reduces the activity or expression of sialidase. In a preferred embodiment, the inhibitor reduces the activity or expression of a sialidase peptide or protein encoded by the neu1 gene.

The sialidase inhibitor may take various forms. Any moiety that inhibits the expression or activity of sialidase can be used in the methods, uses and compositions of the present invention. For example, the inhibitor may be a peptide or protein, a small molecule inhibitor, an inhibitor nucleic acid or a mutant gene or protein.

Some sialidase inhibitors are known to inactivate microbial (bacterial or viral) sialidases associated with infectious diseases. Since there are conserved active site residues between human sialidase, other mammalian and non-mammalian sialidases and microbial sialidases, any inhibitors that affect these sites can be used as novel agents for the treatment of high LDL cholesterol and/or cardiovascular disease in humans.

In a preferred embodiment, the sialidase inhibitor is selected from the group consisting of ADDN (Neu5Ac2en, N-Acetyl-2,3-dehydro-2-deoxyneuraminic acid), 4-amino-Neu5Ac2en (5-acetylamino-2,6-anhydro-4-amino-3,4,5-trideoxy-D-glycerol-D-galacto-non-2-enoic acid), 4-guanidino-NeuSAc2en (5-acetylamino-2,6-anhydro-4-guanidino-3,4,5-trideoxy-D-glycerol-D-galacto-non-2-enoic acid) (Woods et al., 1993) and the like. It is clearly apparent, however, that any molecule having an effect on desialylation is encompassed.

In one preferred embodiment, the inhibitor is a nucleic acid. The nucleic acid may be a nucleic acid encoding a peptide or protein capable of inhibiting sialidase activity. In another embodiment, the nucleic acid is or encodes an anti-sense sequence. In another embodiment, the nucleic add is or encodes a short interfering RNA (RNAi) or a precursor that can be converted to a short interfering RNA. In yet another embodiment, the nucleic acid is a catalytic RNA capable of interfering with expression or abundance or activity of the sialidase enzyme.

In a further aspect of the invention, the use of at least one sialidase inhibitor for the manufacture of a medicament for the treatment of high LDL cholesterol, cardiovascular disease or other metabolic syndrome disorders is provided.

The present invention also provides an animal model in which the neu1 sialidase gene is knocked out. In a further animal model the neu1 gene is knocked out in a mouse have an ApoE−/− genotype.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 is a photomicrograph illustrating the effect of sialidase deficiency on atherosclerosis;

FIG. 2 is a panel of photomicrographs illustrating arterial lesions in apoE knockout and B6SM/apoE knockout mice;

FIG. 3 is a graphical illustration of the quantitation of sizes of atherosclerotic plaques in cross sections of the aortic sinus from male and female apoE knockout and B6SM/apoE knockout mice;

FIG. 4 illustrates graphically the decreased rate of secretion of triglycerides into blood plasma in sialidase deficient B6SM mice compared to control mice;

FIG. 5 illustrates graphically the lipoprotein cholesterol profiles of fat-fed LDL receptor KO mice transplanted with bone marrow from sialidase-deficient or control donors;

FIG. 6 shows atherosclerosis in fat-fed LDL receptor KO mice transplanted with bone marrow from sialidase-deficient or control donors;

FIG. 7 is a graphical representation of the effect of the sialidase inhibitor ADDN on secretion of the pro-inflammatory cytokine IL-6 by macrophages in cell culture;

FIG. 8 is a bar graph illustrating the effect of treatment of apoE KO mice with the sialidase inhibitor ADDN on serum glucose and serum and lipoprotein cholesterol levels; and

FIG. 9 illustrates a morphometric evaluation of the effect of the sialidase inhibitor on atherosclerosis in apoE KO mice.

### DETAILED DESCRIPTION

The invention provides a method for treating a mammalian subject having a condition associated with metabolic syndrome. In particular, a novel method of treating high LDL cholesterol, high triglycerides and associated diseases such as cardiovascular disease and diabetes, by inhibiting sialidase activity is provided. Various types of sialidase inhibitors are useful in the practice of the invention.

The method comprises administering to a subject an amount of an agent that inhibits sialidase activity that is effective to treat or prevent a disorder such as atherosclerosis, diabetes, and hyperlipidemia. The method also encompasses administering a sialidase inhibitor to modulate LDL-C and triglyceride levels.

The term “sialidase inhibitor” is used herein to refer to any moiety that blocks, stops, inhibits and/or suppresses the activity of a sialidase enzyme or the expression of a sialidase peptide or protein from a nucleic acid. Inhibitors useful in the present invention include, but are not limited to peptides, proteins and small molecules that inhibit sialidase activity as well as nucleic acids encoding such inhibitors. The inhibitor may be natural, semi-synthetic, or synthetic. Examples of sialidase inhibitors are disclosed in U.S. Pat. Nos. 5,631,283 and 6,066,323. Nucleic acid molecules such as antisense oligonucleotides, short interfering RNA molecules and catalytic nucleic acids are also useful. In addition, antibodies or antibody fragments that interfere with sialidase activity can be used as sialidase inhibitors. The present invention provides for new uses for sialidase inhibitors.

The agent can be administered by any convenient route. Preferably the agent is administered orally. However, other routes that can be used in accordance with the invention include intravenous, subcutaneous, intramuscular, intraperitoneal and mucosal administration. The compounds can also be delivered through the skin. For example, a patch may be used.

Both human and non-human subject may be treated in accordance with the methods of the invention. The optimal dose can be determined by taking into consideration factors such as the weight and health of the subject and the formulation of the agent.

The sialidase inhibitor compounds suitable for use in accordance with any aspect of the present invention, their pharmaceutically acceptable salts, and pharmaceutically acceptable solvates of either entity can be administered alone or in combination with a suitable pharmaceutical excipient, diluent or carrier.

The sialidase inhibitor compounds or salts or solvates are preferably administered orally in the form of tablets, capsules, gels, films, ovules, elixirs, solutions or suspensions, which may contain flavouring or colouring agents. The compositions may be formulated for immediate-, delayed-, modified-, sustained-, dual-, controlled-release or pulsatile delivery applications.

The sialidase inhibitor compounds suitable for use in accordance with the present invention can also be administered parenterally, for example, intracavernosally, intravenously, intra-arterially, intraperitoneally, intrathecally, intraventricularly, intraurethrally intrasternally, intracranially, intramuscularly or subcutaneously, or they may be administered by infusion or needle-free techniques. For parenteral administration, a sterile aqueous solution containing the inhibitor compound may contain other substances such as salts or glucose to make the solution isotonic with blood.

The daily dosage of the sialidase inhibitor compounds for use in the present invention will be determined based on the severity of the disorder and patient specific factors such as age, weight, etc. For the treatment of various aspects of the Metabolic Syndrome the dosage may by via single dose, divided daily dose, multiple daily dose, acute dosing or continuous (chronic) daily dosing for a specified period.

The inhibitor may be administered alone or in combination with other therapeutic agents. For example, the sialidase inhibitor may be administered together or sequentially with a therapueitc agent such as acyl CoA:cholesterol acyl transferase inhibitor, an apolipoprotein free acceptor, a statin, a resin or bile acid sequestrant, niacin, a liver X receptor agonist, a Ca2+ antagonist or a modulator of peroxisome proliferator-activated receptors. The inhibitor may be provided in combination with any other therapeutic compound that is useful for the treatment of a metabolic syndrome disorder. A pharmaceutical composition of the invention may combine an inhibitor and an additional therapeutic agent in combination.

The compounds and compositions of the invention are useful in the treatment and/or prevention of a variety of disorders including, but not limited to, insulin resistance syndrome, diabetes, hyperlipidemia, fatty liver disease, cachexia, obesity, atherosclerosis, and arterioscerlosis.

Exemplary sialidase inhibitors for use in the invention include, but are not limited to, ADDN, Neu5Ac2en, 9-azido-Neu5Ac2en, 9-NANP-Neu5Ac2en and the like. Anitbodies that interfere with sialidse activity can also be used as inhibitors. Nucleic acid inhibitors are also useful in the invention.

The efficacy of the use of a sialidase inhibitor as an agent for the treatment of high LDL cholesterol or triglycerides and cardiovascular disease, diabetes and related diseases was demonstrated using animal models that are well accepted as models of human cardiovascular disease and high LDL cholesterol. The LDL receptor KO/bone marrow transplantation model, is a genetic model demonstrating the effect of reduced sialidase expression on LDL cholesterol levels and atherosclerotic plaque development in animals fed a high fat diet. The B6SM/apo E knockout model is a genetic model demonstrating the effect of reducing sialidase expression on spontaneous atherosclerosis. The apoE knockout model is a therapeutic model demonstrating the effect of administering a sialidase inhibitor on spontaneous atherosclerosis. The following description of these models relate to preferred embodiments demonstrating the efficacy and utility of the invention and does not limit the scope of the invention.

In one aspect, the present invention provides a novel animal model for the study of sialidase activity. An inbred mouse strain, SM/J, has a relatively high susceptibility for aortic atherosclerosis. The SM/J strain potentially harbors mutations in several genes, including a sialidase gene, which may contribute to its complex phenotype. In order to demonstrate the contribution of sialidase deficiency to the atherosclerotic phenotype of the SM/J strain, the sialidase mutation was isolated from the SM/J mouse background by backcrossing onto the unrelated C57BI/6 inbred genetic background to generate the B6.5M strain of mice. The effects of neu 1 sialidase deficiency could therefore be studied in the absence of other mutations in the SM/J strain. The effects of sialidase deficiency alone or in combination with other factors influencing cardiovascular disease, including lipoprotein cholesterol metabolism, diabetes and atherosclerosis were analyzed.

The ApoE knockout (KO) mouse model was used as a model of spontaneous atherosclerosis. These mice lack a functional gene for apolipoprotein E, a component of a variety of lipoproteins. These mice exhibit increased levels of cholesterol associated with LDL, larger sized lipoproteins, decreased levels of cholesterol associated with HDL and a tendency to develop atherosclerosis spontaneously when fed diets with normal fat content and to an increased extent when fed high fat diets.

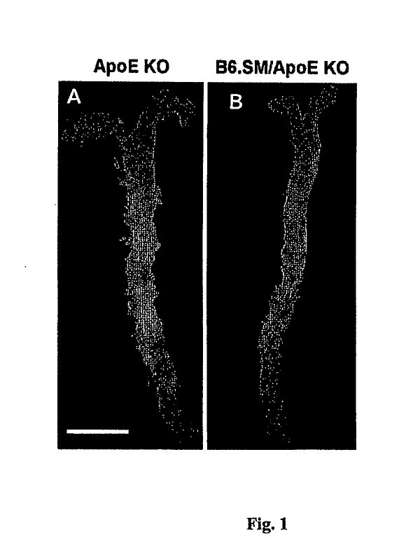

In a further aspect of the invention, ApoE KO mice that were also deficient in neu1 sialidase gene expression (B6SM/apoE KO) were generated by crossing B6SM and apoE KO mice through two generations. Atherosclerosis in this novel murine model was compared to that in control ApoE KO mice with normal sialidase gene expression. FIG. 1 shows the extent of lipid-rich atherosclerosis in representative aortas from an ApoE KO and a B6SM/apoE KO mouse. The results demonstrate a significantly lower amount of atherosclerotic plaque covering the inner aorta in the B6SM/apoE KO mice than in the aorta from control apoE KO mice.

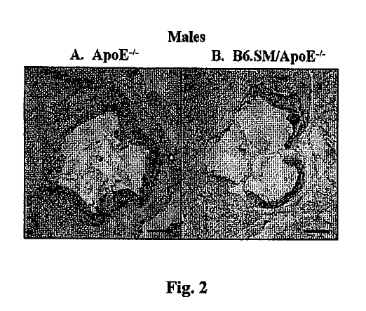

This effect is further demonstrated in FIG. 2. FIG. 2 shows cross sections through the aortic root of the aortic sinus from B6SM/apoE KO and control apoE KO mice. Sections through the aortic root were stained for lipid with Oil red O and counterstained for nuclei with hematoxylin. The micrographs of FIG. 2 demonstrate that there is a reduction in atherosclerotic plaques in the sialidase deficient mice.

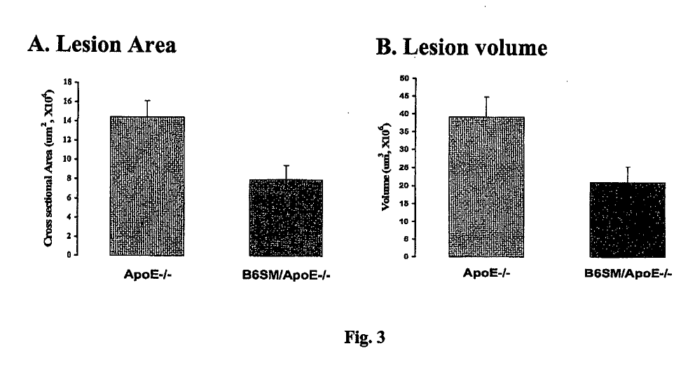

The cross sectional areas of atherosclerotic plaque were measured for each section and the approximate volume of atherosclerotic plaque in a section of the aortic sinus was determined. FIG. 3A shows the average cross sectional area of atherosclerosis at the aortic root and FIG. 3B shows the average volumes of atherosclerotic plaque in a segment of the aortic sinus, demonstrating reduced atherosclerosis in sialidase deficient B6SM/apoE KO mice relative to control apoE KO mice. The results demonstrate, for the first time, that suppression of neu1 sialidase gene expression with the associated decrease in enzyme activity, can suppress the development of atherosclerosis.

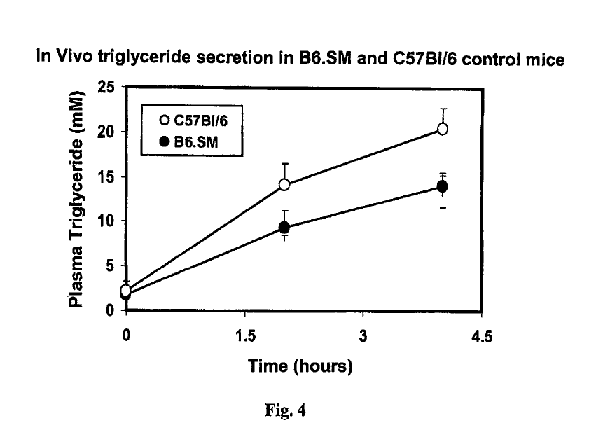

Inhibition of sialidase as a therapeutic approach for metabolic syndrome disorders was further validated by measuring the rate of secretion of triglycerides into plasma in fasted sialidase-deficient B6.5M and control C57BI/6 mice. The results are shown in FIG. 4. Mice were injected intravenously with the chemical Triton-WR1339 to block clearance of newly synthesized and secreted triglyceride-rich lipoproteins, allowing them to accumulate in plasma according to the method of Kuipers et al, 1997. Triglyceride concentrations in plasma were measured at times 0, 2 and 4 hours after Triton WR1339 injection. The results shown in FIG. 4 clearly demonstrate that there is lower triglyceride secretion into blood plasma in sialidase-deficient B6.5M mice than in control C57BI/6 mice. Thus, inhibition of sialidase activity is associated with decreased blood triglyceride levels.

To further demonstrate the therapeutic efficacy of sialidase inhibitors, the LDL receptor KO mouse model of diet-induced atherosclerosis was used. LDL receptor KO mice lack a functional gene for the LDL receptor, resulting in increased blood LDL-C levels, which are further increased when the mice are fed a high fat diet. These mice develop extensive atherosclerosis when fed diets rich in fat.

To generate LDL receptor KO mice with reduced sialidase, a bone marrow transplantation approach was utilized. Briefly, bone marrow from either B6.5M (suppressed sialidase expression) or control C57B[/6 mice (normal sialidase expression) was transplanted into lethally irradiated LDL receptor KO mice. The resulting mice lacked the LDL receptor in most tissues, making them susceptible to diet induced atherosclerosis. Bone marrow derived blood cells, including cells of the immune system (monocytes/macrophages, dendritic cells, T-lymphocytes, etc) either had a normal or mutant sialidase gene, depending on the bone marrow donor. Using this model, it was demonstrated that decreased sialidase expression and therefore decreased sialidase activity, resulted in reduced levels of cholesterol associated with low density lipoproteins (LDL-C) and reduced the development of atheroscierosis. This indicates that inhibition of the neu 1 sialidase in blood cells can be a beneficial therapeutic strategy for treatment of high triglycerides, hypercholesterolemia (i.e. for LDL lowering), cardiovascular disease and associated diseases including diabetes.

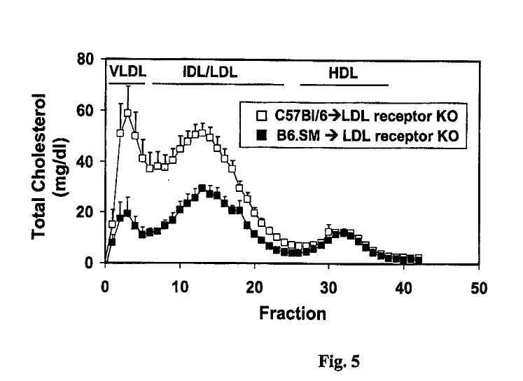

FIG. 5 shows the lipoprotein cholesterol profiles from fat-fed LDL receptor KO mice transplanted with either control C57BI/6 or sialidase deficient B6.5M bone marrow. Mice with reduced sialidase expression (transplanted with bone marrow from B6.5M donors) had substantially reduced levels of total lipoprotein cholesterol (˜50% reduction). This was the result of reduced levels of cholesterol associated with the atherogenic very low-density lipoproteins, VLDL, (67% reduction) and intermediate density lipoproteins (IDL) and LDL (46% reduction). In contrast HDL cholesterol levels were only reduced slightly and the difference was not statistically significant.

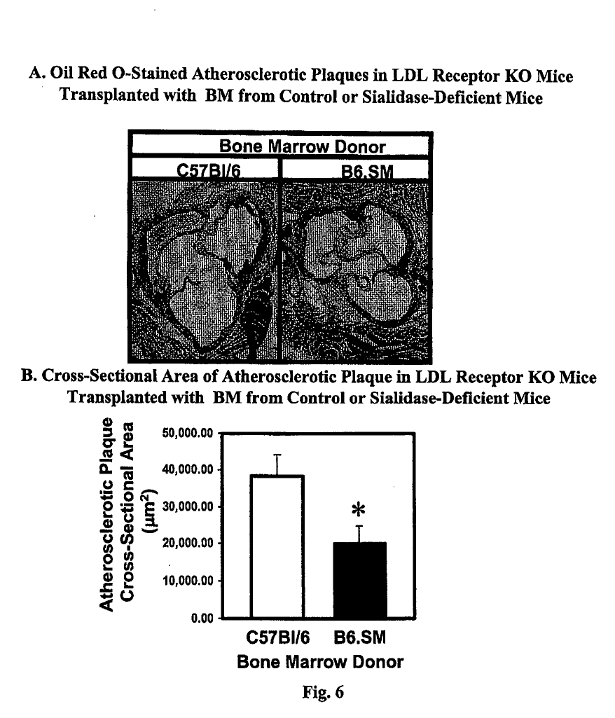

Atherosclerosis in the aortic sinus of the high fat diet-fed LDL receptor KO mice transplanted with either control C57BI/6 or sialidase deficient B6.5M bone marrow was analyzed using a standard morphometric approach. FIG. 6A illustrates atherosclerotic plaques in a cross-section of the aortic sinuses of representative LDL receptor KO mice that received bone marrow transplanted from either control C57B16 or sialidase deficient B6SM donors. FIG. 6B shows the average atherosclerotic plaque cross sectional area measured for the transplanted mice with either normal or reduced sialidase expression. Mice with reduced sialidase expression in bone marrow derived cells had a substantial (about 50%) reduction in diet-induced atherosclerosis. This data demonstrates for the first time that suppression of sialidase expression and therefore activity in bone marrow derived blood cells can reduce levels of cholesterol associated with atherogenic lipoproteins (VLDL, IDL, LDL) but not protective lipoproteins (HDL) and can suppress the development of atherosclerosis. Furthermore, the results indicate that inhibition of sialidase activity is an important therapeutic strategy for lowering LDL cholesterol and triglyceride levels and for prevention or treatment of cardiovascular disease.

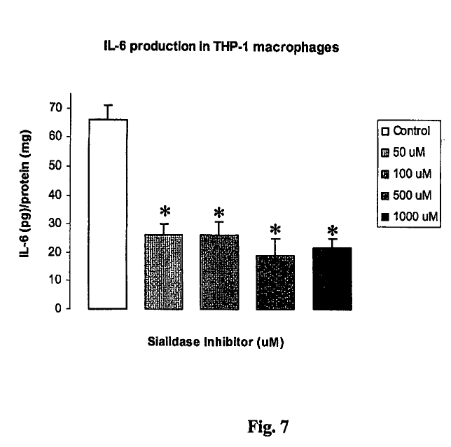

The observation that reduced sialidase activity in bone marrow-derived blood cells suppresses atherosclerosis suggests the involvement of sialidase in controlling one or more pathways involved in inflammation. The effects of a sialidase inhibitor, N-acetyl-2,3-dehydro-2-deoxyneuraminic acid (ADDN), on the production of the inflammatory cytokine interleukin-6 (IL-6) by differentiated human THP-1 macrophages in culture was measured. The results shown in FIG. 7, demonstrate that inhibition of sialidase with ADDN results in reduced production of IL-6. This suggests that suppression of sialidase activity may suppress atherosclerosis by reducing inflammation in addition to reducing LDL-cholesterol and plasma triglycerides. This demonstrates the beneficial effects of suppressing sialidase activity on cardiovascular disease, the metabolic syndrome and diabetes, and also for other diseases involving inflammation.

In further support for the use of a sialidase inhibitor as a therapeutic for metabolic syndrome disorders, an exemplary inhibitor was demonstrated to be effective in lowering LDL-C and reducing glucose levels using the apoE KO mouse model.

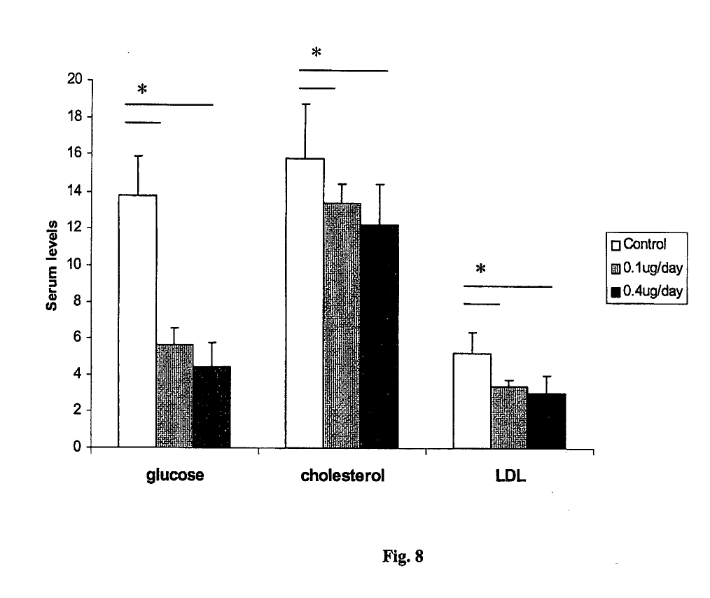

The effect of an inhibitor of the neu 1 sialidase, N-acetyl-2,3-dehydro-2-deoxyneuraminic acid (ADDN), on serum and lipoprotein cholesterol levels, serum glucose and atherosclerosis in apolipoprotein E KO mice was determined. Mice that received the sialidase inhibitor, ADDN, had significantly less serum total cholesterol, LDL cholesterol and HDL cholesterol than did the control mice. ApoE knockout mice also normally develop hyperglycemia (high serum glucose). Mice that were treated with ADDN had significantly decreased levels of serum glucose than did the control mice. These results are shown in FIG. 8. Consistent with the results from genetic suppression of sialidase, (FIG. 5 and Table 1), the reduction in LDL-cholesterol was greater than the slight reduction in HDL cholesterol.

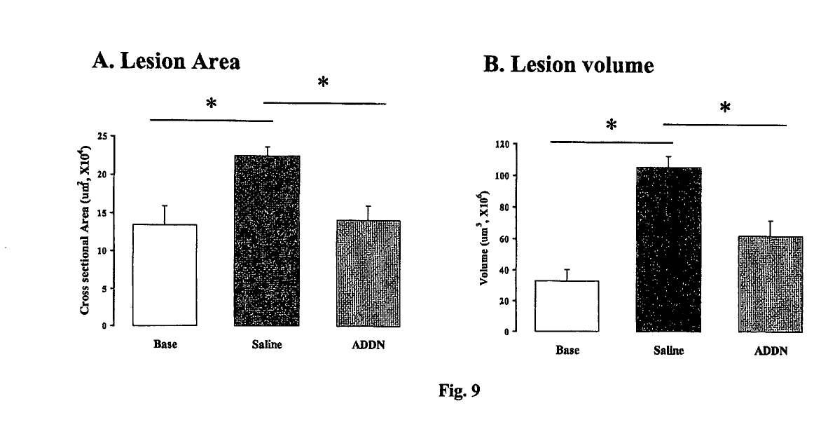

FIG. 9 further illustrates that the ADDN treatment suppressed atherosclerosis development in apoE knockout animals.

Suppression of sialidase activity results in decreased LDL-C levels, and decreased blood glucose. The results indicate that sialidase inhibitors are useful in lowering LDL cholesterol and in treatment of diabetes. Furthermore, suppression of sialidase activity reduces diet-induced and spontaneous atherosclerosis in mice. The use of a sialidase inhibitor in accordance with the present invention has tremendous potential for the treatment of high LDL cholesterol or triglycerides, cardiovascular disease and diabetes, and other diseases associated with the Metabolic Syndrome.

The above disclosure generally describes the present invention. It is believed that one of ordinary skill in the art can, using the preceding description, make and use the compositions and practice the methods of the present invention. A more complete understanding can be obtained by reference to the following specific examples. These examples are described solely to illustrate preferred embodiments of the present invention and are not intended to limit the scope of the invention. Changes in form and substitution of equivalents are contemplated as circumstances may suggest or render expedient. Other generic configurations will be apparent to one skilled in the art. All journal articles and other documents such as patents or patent applications referred to herein are hereby incorporated by reference.

### EXAMPLES

Although specific terms have been used in these examples, such terms are intended in a descriptive sense and not for purposes of limitation. Methods of molecular biology, biochemistry and chemistry referred to but not explicitly described in the disclosure and these examples are reported in the scientific literature and are well known to those skilled in the art.

### Example 1

### Mice

All procedures involving mice were carried out in accordance with institutional and Canadian Council on Animal Care guidelines. C57BI/6, SM/J, apo E KO and LDL receptor KO mice (on a C57BI/6 background) were from the Jackson Laboratories. B6.5M mice were generated by backcrossing SM/J mice with C57BI/6 mice, selecting for the mutant sialidase allele in offspring. All mice had free access to food and water unless otherwise indicated.

### Example 2

### Bone Marrow Transplantation

Bone marrow transplantation was carried out as described previously (Covey et al, 2003). Briefly, male LDL receptor KO recipients were exposed to total body dose of 12 Gy of ¹³⁷Cs-gamma irradiation, administered in two portions (8 Gy and 4 Gy) separated by 3 hrs. Bone marrow was collected from the tibias and femurs of either control C57BI/6 or sialidase deficient B6.5M mice that had been euthanized by asphyxiation with CO₂. Irradiated recipient mice were anesthetized with 2.5% avertin in saline (administered intraperitoneally at ˜0.1 mil/10 g body weight), and 6×10⁶ bone marrow cells were injected intravenously. Mice were maintained after transplantation on antibiotics (Covey et al 2003). Four weeks after transplantation, blood was collected and blood cell DNA was prepared. Donor bone marrow engraftment was assessed by PCR detection of the wild type (donor derived) and mutant (recipient derived) LDL receptor alleles. All mice used in the study showed complete donor cell engraftment.

FIG. 1 shows the effect of sialidase deficiency on atherosclerotic lesion development. Images of plaque-covered luminal surface of aortas isolated from representative male ApoE−/− (A) and B6SM/ApoE−/− (B) animals. Formalin-fixed vessels were stained for lipid rich atherosclerotic plaques with Sudan IV, cut open longitudinally and mounted individually on glass slides. Atherosderotic plaques are visible as red deposits. Scale bar-5 mm.

FIG. 2 shows pathological evaluations of arterial lesions in ApoE−/− and B6.5M/ApoE−/− mice. Cross-sections of the aortic sinus from male ApoE−/− (A) and male B6.5M/ApoE−/− (B) mice. Mice were anesthetized with ketamine/rompun. The abdominal and thoracic cavities were opened and the heart was perfused with PBS (4° C.) through the left ventricle of the heart (drainage via the right atrium). The heart was removed and placed in Krebs Henseleit Solution. After 30 min, the heart was placed in 10% formaldehyde at 4° C. After 24 hours, the heart was placed in PBS. After another 24 hours, the heart was placed in 30% sucrose with PBS. Hearts were frozen in Cryomatrix (Shandon Corp) and serial 10 μm sections were collected. Sections were stained with Oil Red 0 for neutral lipid and hematoxylin for nuclei. Scale bar=200 μm.

FIG. 3 shows a morphometric evaluation of atherosclerotic lesion area (A) and volume (B) in the aortic sinus of ApoE−/− and B6.5M/ApoE−/− mice. Sections were prepared and stained. Atherosclerotic lesion areas were quantified as the total cross sectional area of atherosclerotic plaque in each section. Panel A shows the average lesion area for the arotic root (corresponding to the sections shown in FIG. 2). Cross sectional areas of lesions in 8 sections spaced 100 μm apart were taken as the average lesion area for the 100 μm stretch of the aortic sinus. The sum of these was taken as the lesion volume (panel B). Results are the means±standard error for male ApoE−/− (n=17) and B6.5M/ApoE−/− (n=15). Student's T-test was used to determine statistical significance (* denotes P<0.05; * denotes P<0.001).

FIG. 4 shows triglyceride secretion into plasma in male sialidase deficient B6SM or control wild type C57BI6 mice. Triton WR1339 interferes with the normally rapid clearance of newly secreted, triglyceride-rich VLDL from plasma, resulting its accumulation. Male mice were fasted overnight and Triton WR 1339 (500 mg/kg body weight; 150 mg/ml in 0.9% NaCl) was injected intravenously via the tail vein. Plasma (50 ml) was collected via the saphenous vein at 0, 2 and 4 hours after injection. TG concentrations in plasma were measured using an enzymatic assay from Wako Diagnostics. Data are the means of measurements from three mice per genotype. The data indicates that triglyceride secretion into plasma is reduced in sialidase-deficient B6.5M mice relative to control C57BI/6 mice.

FIG. 5 shows the lipoprotein cholesterol profiles of fat-fed LDL receptor KO rice transplanted with bone marrow from sialidase-deficient or control donors. Male LDL receptor KO mice were lethally irradiated (12 Gy) and reconstituted with bone marrow (BM) prepared from the tibias and femurs of donor sialidase-deficient B6.5M mice (filled squares) or control C57BI/6 mice (open squares). One month following transplantation, BM engraftment was tested by PCR genotyping of blood cell DNA, and mice reconstituted with donor-derived bone marrow were fed a high fat, Western-type diet for 6 weeks. Plasma was collected after an overnight fast and lipoproteins were size-fractionated on a Superose 6 HR 10/30 column. The amount of cholesterol was determined in each fraction and expressed as the concentration in plasma. The positions at which human VLDL, IDL/DL and HDL elute from the column are indicated. Each profile is the average of profiles from independent mice (n=8 for C57BV6 donors and n=4 for B6.5M donors). Error bars represent the standard error of the mean.

FIG. 6 shows atherosclerosis in mice fat-fed LDL receptor KO rice transplanted with bone marrow from sialidase-deficient or control donors. Male LDL receptor KO mice were transplanted with bone marrow from donor sialidase-deficient B6.5M mice (right panel in A, filled column in B) or control C57BI/6 mice (left panel in A, open column in B) and fed a high fat Western type diet. Atherosclerosis was measured in oil red O-stained frozen sections of the aortic sinus. Panel A shows representative sections from mice reconstituted with bone marrow from control C57BI/6 (left) or sialidase-deficient B6.5M donors (right). B. The amount of atherosclerosis was measured as the mean cross sectional area of plaque. Error bars represent the standard error of the mean (n=9 or 11 for C57BI/6 or B6.5M donors; P<0.01).

FIG. 7 illustrates that the inhibition of sialidase activity suppresses production of the pro-atherogenic cytokine IL-6 in differentiated THP-1 macrophages. Differentiated THP-1 macrophages were incubated for 3 days with the varying concentrations of the sialidase inhibitor ADDN. Levels of the cytokine IL-6 in samples of the cell culture supernatant were quantified by ELISA. Data are presented as the mean±standard deviation of triplicate experimental groups. (* denotes significant difference, P<0.001).

FIG. 8 shows a comparison of serum glucose, total cholesterol and LDL levels for ApoE knockout mice treated for one week with daily injections of ADDN (N-Acetyl-2,3-dehydro-2-deoxyneuraminic add) at the indicated doses. Control mice received daily injections of saline. Data reveal significant reductions in serum glucose levels, and total and LDL cholesterol. P<0.01.

FIG. 9 illustrate a morphometric evaluation of atherosclerotic lesion area (A) and volume (B) in the aortic sinus of male ApoE−/− mice treated for 6 weeks with the sialic acid inhibitor ADDN (N-Acetyl-2,3-dehydro-2-deoxyneuraminic acid). Mice aged 7 months were treated for 6 weeks with either saline or the sialic acid inhibitor ADDN. (n=8 in each case). A third group of mice was euthanized at the beginning of the experiment so that the level of atherosclerosis at the time that treatment was initiated could be measured (Base, n=9). Atherosclerotic lesion cross sectional areas in the aortic root (A) and lesion volumes (B) were measured. Values are the averages ±standard error of the mean. Lesion progression was reduced in ADDN-treated mice compared to saline controls. Student's T-test was used to determine statistical significance (* denotes P<0.001).

As illustrated in FIG. 6, male LDL receptor KO mice transplanted with bone marrow from donor sialidase-deficient B6.5M mice (right panel in A, filled column in B) or control C57BI/6 mice (left panel in A, open column in B) and fed a high fat Western type diet were assessed for atherosclerosis. Atherosclerosis was measured in oil red O-stained frozen sections of the aortic sinus. Panel A shows representative sections from mice reconstituted with bone marrow from control C57BI/6 (left) or sialidase-deficient B6.5M donors (right). Panel B illustrates the amount of atherosclerosis as the mean cross sectional area of plaque. Error bars represent the standard error of the mean (n=9 or 11 for C57BI/6 or B6.5M donors; P=0.01).

### Example 3

### Plasma and Lipoprotein Cholesterol Analysis

Plasma was prepared from heparinized blood collected by cardiac puncture (Covey et al 2003). Serum was collected from blood using serum separators (Becton Dickenson). Serum was submitted to the Clinical Diagnostic Laboratory at the McMaster University Medical Centre for analyses of standard metabolic parameters including total cholesterol, LDL cholesterol and glucose. The results are shown in FIG. 8. Lipoproteins from plasma were separated by size by fast protein liquid chromatography (FPLC) using an AKTA system (Amersham Biosciences, Inc.) with a Superose 6 HR 10/30 column, as described previously (Covey et al 2003). The total cholesterol content in each fraction was assayed using an enzymatic assay kit from Thermo DMA. Lipoproteins (VLDL, LDL and HDL) from human plasma were run as controls to calibrate the column. The results are shown in FIG. 5.

The amount of cholesterol was determined in each fraction and expressed as the concentration in plasma. The positions at which human VLDL, IDL/DL and HDL elute from the column are indicated. Each profile is the average of profiles from independent mice (n=8 for C57BI/6 donors and n=4 for B6.5M donors). Error bars represent the standard error of the mean.

Lipoprotein total cholesterol, and cholesterol associated with VLDL, IDL/LDL and HDL sized fractions was determined from the profiles of individual mice (see FIG. 5). The cholesterol levels are shown in Table 1.

| | Total | VLDL | IDL/LDL | HDL |
|---|---|---|---|---|
| | Cholesterol | Cholesterol | Cholesterol | Cholesterol |
| BM Donor | (mg/dL) | (mg/dL) | (mg/dL) | (mg/dL) |
| Control | 980 ± 99 | 328 ± 54 | 535 ± 37 | 90 ± 5 |
| C57B1/6 | | | | |
| Sialidase | 494 ± 45* | 109 ± 18* | 289 ± 34* | 79 ± 11 |
| Deficient | | | | |
| B6.SM | | | | |

Table 1 shows plasma total cholesterol, and cholesterol associated with VLDL-, IDL/DL- and HDL-sized lipoprotein particles. Values, determined from the data represented in FIG. 5 are the averages ±standard errors of n=8 for C57BI/6 donors and n=4 for B6.5M donors. * indicates P<0.002 compared to mice receiving control C57BI/6 bone marrow.

### Example 4

### Diet Induced and Spontaneous Atherosclerosis

For diet-induced atherosclerosis, mice were fed (beginning four weeks after transplantation) with a high fat, Western-type diet (Covey et al 2003) obtained from Dyets, Inc. (Bethlehem Pa.). After 6 weeks of high fat diet feeding, mice were fasted overnight and euthanized by avertin anesthetic overdose. For analysis of spontaneous atherosclerosis, mice were fed a control mouse diet containing normal levels of fats, and were euthanized at 7 months of age. Blood was collected by cardiac puncture and plasma was prepared as described previously (Covey et al 2003). Mice were perfused with saline to clear vessels of blood. Hearts were removed, incubated in Krebs Henseleit Solution for 30 min and then fixed in 10% formalin overnight, rinsed in saline, incubated in 30% sucrose and frozen in Cryomatrix (Shandon Inc) in a 2-methylbutane/dry ice bath. Ten-micrometer thick tissue sections were collected using a Shandon Cryomicrotome. Sections corresponding to the aortic root region in the vicinity of the aortic valve leaflets were stained for lipid with Oil Red 0 and counterstained for nuclei using Mayer's hematoxylin (stains were from Sigma Chemical Company Inc., St. Louis, Mo.). Images were captured using a Zeiss Axiovert 200 M inverted microscope fitted with a 5× objective. Atherosclerotic plaque cross sectional area was measured by morphometry using Axiovision software (Carl Zeiss Canada, Inc). Exemplary results are shown in FIGS. 2 and 6. A total of eight sections lying at 0.1 mm intervals along the aortic sinus were analyzed for each mouse. The cross sectional area of atherosclerosis in each section was taken as the average cross sectional area for the corresponding 0.1 mm portion of the aortic sinus centering on the position of the section. The volume of atherosclerotic plaque was therefore calculated as the sum of the volumes determined for each 0.1 mm portion of the aortic sinus. Exemplary results are shown in FIGS. 3 and 9.

### Example 5

### Treatment with the Sialidase Inhibitor N-Acetyl-2,3-dehydro-2-deoxyneuraminic Acid (ADDN)

The inhibitor, N-Acetyl-2,3-dehydro-2-deoxyneuraminic acid (ADDN), was obtained from Sigma Chemical Company (St. Louis, Mo.). ApoE KO mice were ˜6 weeks of age at the beginning of the study. The mice were randomized into 2 groups of 6. The control group received daily i.p injections of 0.9% saline (0.1 ml/day for 13 days). The experimental group received daily i.p. injections of 0.9% saline (0.1 ml per day) containing either 0.1 or 0.4 ug ADDN for 7 days. Serum was collected on day 7 and serum glucose, total cholesterol and LDL levels were measured. The results are shown in FIG. 8.

### Example 6

### The Effect of Treatment of apoE KO Mice with the Sialidase Inhibitor ADDN on Atherosclerosis

For this experiment, mice were 7 months of age and divided into three groups. The first group (17 mice) was euthanized immediately and atherosclerosis was assessed to provide a baseline atherosclerosis measurement. The other two groups (8 mice each) were treated with either 0.9% saline (control) or 0.9% saline containing 0.284 μg/ml ADDN using mini-osmotic pumps to deliver 5.28 μl per day (flow rate was 0.22 micro-l/hr) so that mice received either 0 (control) or 1.5 micro-g/day ADDN. Treatment was for a total of 6 weeks, at which time mice were euthanized and atherosclerosis was assessed. The results are shown in FIG. 9.

Atherosclerotic plaque cross sectional area (FIG. 9A) and volume (FIG. 9B) increased in control, saline treated mice from the baseline level, over the 6-weeks of saline treatment. In contrast, the growth of plaques from the baseline level was substantially inhibited in mice treated with ADDN over the 6-week treatment period. This demonstrates for the first time that the chemical inhibition of sialidase activity can suppress the development of atherosclerosis.

All citations are hereby incorporated by reference.

The present invention has been described with regard to one or more embodiments. However, it will be apparent to persons skilled in the art that a number of variations and modifications can be made without departing from the scope of the invention as defined in the claims.

### REFERENCES

Anunciado, R. V., Nishimura, M., Mori, M., Ishikawa, A., Tanaka, S., Horio, F., Ohno, T., Namikawa, T., 2001. Quantitative trait loci for body weight in the intercross between SM/J and A/J mice. Exp. Anim 50, 319-324.
Bhatia-Gaur, R., Donjacour, A. A., Sciavolino, P. J., Kim, M., Desai, N., Young, P., Norton, C. R., Gridley, T., Cardiff, R. D., Cunha, G. R., Abate-Shen, C., Shen, M. M., 1999. Roles for Nkx3.1 in prostate development and cancer. Genes Dev. 13, 966-977.
Bieberich, C. J., Fujita, K., He, W. W., Jay, G., 1996. Prostate-specific and androgen-dependent expression of a novel homeobox gene. J. Biol. Chem. 271, 31779-31782.
Bonten, E., van der, S. A., Fomerod, M., Grosveld, G., d'Azo, A., 1996. Characterization of human lysosomal neuraminidase defines the molecular basis of the metabolic storage disorder sialidosis. Genes Dev. 10, 3156-3169.
Bonten, E. J., Arts, W. F., Beck, M., Covanis, A., Donati, M. A., Parini, R., Zammarchi, E., d'Azzo, A., 2000. Novel mutations in lysosomal neuraminidase identify functional domains and determine clinical severity in sialidosis. Hum. Mol. Genet. 9, 2715-2725.
Carrillo, M. B., Milner, C. M., Ball, S. T., Snoek, M., Campbell, R. D., 1997. Cloning and characterization of a sialidase from the murine histocompatibility-2 complex: low levels of mRNA and a single amino acid mutation are responsible for reduced sialidase activity in mice carrying the Neu1(a) allele. Glycobiology 7, 975-986.
Champigny, M. J., Johnson, M., Igdoura, S. A., 2003. Characterization of the mouse lysosomal sialidase promoter. Gene 319, 177-187.
Chen, X. P., Ding, X., Daynes, R. A., 2000. Ganglioside control over IL-4 priming and cytokine production in activated T cells. Cytokine 12, 972-985.
Chen, X. P., Enioutina, E. Y., Daynes, R. A., 1997. The control of IL4 gene expression in activated murine T lymphocytes: a novel role for neu-1 sialidase. J. Immunol. 158, 3070-3080.
Cheverud, J. M., Vaughn, T. T., Pletscher, L. S., Peripato, A. C., Adams, E. S., Erikson, C. F., King-Ellison, K. J., 2001. Genetic architecture of adiposity in the cross of LG/J and SM/J inbred mice. Mamm. Genome 12, 3-12.
Clark, E. A., Engel, D., Windsor, N. T., 1981a. Immune responsiveness of SM/J mice: hyper NK cell activity mediated by NK 1+ Qa 5− cells. J. Immunol. 127, 2391-2395.
Collard, J. G., Schijven, J. F., Bikker, A., La Riviere, G., Bolscher, J. G., Roos, E., 1986. Cell surface sialic acid and the invasive and metastatic potential of T-cell hybridomas. Cancer Res. 46, 3521-3527.
Cornelli, E. M., Amado, M., Lustig, S. R., Paulson, J. C., 2003. Identification and expression of Neu4, a novel murine sialidase. Gene 321, 155-161.
Constant, S. L., Bottomly, K., 1997. Induction of Th1 and Th2 CD4+ T cell responses: the alternative approaches. Annu. Rev. Immunol. 15, 297-322.
Covey S D, Krieger M, Wang W, Penman M, Trigatti BL., 2003. Scavenger Receptor Class B Type I-Mediated Protection Against Atherosclerosis in LDL Receptor-Negative Mice Involves Its Expression in Bone Marrow-Derived Cells. Arterioscier Thromb Vasc Biol. 23, 1.589-1594.
Cuff C, Kothapalli D, et al. (2001) The adhesion receptor CD44 promotes atherosclerosis by mediating inflammatory cell recruitment and vascular cell activation. J. Clinc. Invest. 108:1031-1040.
Cullen, P., et al., 1997. Lipoproteins and cardiovascular risk-from genetics to CHD prevention. J Atheroscler Thromb, 4: 51-58.
Daniel, W. L., Womack, J. E., Henthom, P. S., 1981. Murine liver arylsulfatase B processing influenced by region on chromosome 17. Biochem. Genet. 19, 211-225.
Dizik, M., Elliott, R. W., 1977. A gene apparently determining the extent of sialylation of lysosomal alpha-mannosidase in mouse liver. Biochem. Genet. 15, 3146.
Engel, D., Clark, E. A., Held, L., Kimball, H., Clagett, J., 1981. Immune responsiveness of SM/J mice. Cellular characteristics and genetic analysis of hyperresponsiveness to B cell mitogens. J. Exp. Med. 154, 726-736.
Ferrari, J., Harris, R., Warner, T. G., 1994. Cloning and expression of a soluble sialidase from Chinese hamster ovary cells: sequence alignment similarities to bacterial sialidases. Glycobiology 4, 367-373.
Figueroa, F., Klein, D., Tewarson, S., Klein, J., 1982a. Evidence for placing the Neu-1 locus within the mouse H-2 complex. J. Immunol. 129, 2089-2093.
Frangioni, J. V., Neel, B. G., 1993. Solubilization and purification of enzymatically active glutathione S-transferase (pGEX) fusion proteins. Anal . Biochem. 210, 179-187.
Gee, K., Kozlowski, M., Kumar, A., 2003. Tumor necrosis factor-alpha induces functionally active hyaluronan-adhesive CD44 by activating sialidase through p38 mitogen-activated protein kinase in lipopolysaccharide-stimulated human monocytic cells. J. Biol. Chem. 278, 37275-37287.
Gordon, D. J. and B. M. Rifkind, 1989. High-density lipoprotein-the clinical implications of recent studies. N Engl J Med, 321, 1311-1316.
Green, R. C., Green, A. G., Simms, M., Pater, A., Robb, J. D., Green, J. S., 2003. Germline hMLH1 promoter mutation in a Newfoundland HNPCC kindred. Clin. Genet. 64, 220-227.
Herbrand, H., Pabst, O., Hill, R., Amold, H. H., 2002. Transcription factors Nkx3.1 and Nkx3.2 (Bapx1) play an overlapping role in scierotomal development of the mouse. Mech. Dev. 117, 217-224.
Hojo, Y., Ikeda, U., Takahashi, M., Shimada, K., 2002. Increased levels of monocyte-related cytokines in patients with unstable angina. Atherosclerosis 161, 403-408.
Igdoura, S. A., Gafuik, C., Mertineit, C., Saberi, F., Pshezhetsky, A. V., Potier, M., Trasler, J. M., Gravel, R. A., 1998. Cloning of the cDNA and gene encoding mouse lysosomal sialidase and correction of sialidase deficiency in human sialidosis and mouse SM/J fibroblasts. Hum. Mol. Genet. 7, 115-121.
Ishibashi S. Brown M S, Goldstein J L, Gerard RD, Hammer RE, Herz J, 1993. Hypercholesterolemia in low density lipoprotein receptor knockout mice and its reversal by adenovirus-mediated gene delivery. J Clin Invest. 92, 883-893.
Itokawa, M., Arai, M., Kato, S., Ogata, Y., Furukawa, A., Haga, S., Ujike, H., Sora, I., Ikeda, K., Yoshikawa, T., 2003. Association between a novel polymorphism in the promoter region of the neuropeptide Y gene and schizophrenia in humans. Neurosci. Lett. 347, 202-204.
Jacob, C. O., Tashman, N. B., 1993. Disruption in the AU motif of the mouse TNF-alpha 3′ UTR correlates with reduced TNF production by macrophages in vitro. Nucleic Acids Res. 21, 2761-2766.
Katoh, S., Miyagi, T., Taniguchi, H., Matsubara, Y., Kadota, J., Tominaga, A., Kincade, P. W., Matsukura, S., Kohno, S., 1999. Cutting edge: an inducible sialidase regulates the hyaluronic acid binding ability of CD44-bearing human monocytes. J. Immunol. 162, 5058-5061.
Kim, D. W., Kempf, H., Chen, R. E., Lassar, A. B., 2003. Characterization of Nkx3.2 DNA binding specificity and its requirement for somitic chondrogenesis. J. Biol. Chem. 278, 27532-27539.
Kim, D. W., Lassar, A. B., 2003. Smad-dependent recruitment of a histone deacetylase/Sin3A complex modulates the bone morphogenetic protein-dependent transcriptional repressor activity of Nkx3.2. Mol. Cell. Biol. 23, 8704-8717.
Klein, D., Klein, J., 1982. Polymorphism of the Apl (Neu-1) locus in the mouse. Immunogenetics 16, 181-184.
Korstanje, R., Eriksson, P., Samnegard, A., Olsson, P. G., Forsman-Semb, K., Sen, S., Churchill, G. A., Rollins, J., Harris, S., Hamsten, A., Paigen, B. 2004. Locating Ath8, a locus for murine atherosclerosis susceptibility and testing several of its candidate genes in mice and humans. Atherosclerosis 177, 443-450.
Kos, L., Chiang, C., Mahon, K. A., 1998. Mediolateral patterning of somites: multiple axial signals, including Sonic hedgehog, regulate Nkx-3.1 expression. Mech. Dev. 70, 25-34.
Kubaszek, A., Pihlajamaki, J., Komarovski, V., Lindi, V., Lindstrom, J., Eriksson, J., Valle, T. T., Hamalainen, H., Ilanne-Parikka, P., Keinanen-Kiukaanniemi, S., Tuomilehto, J., Uusitupa, M., Laakso, M., 2003. Promoter polymorphisms of the TNF-alpha (G-308A) and IL-6 (C-174G) genes predict the conversion from impaired glucose tolerance to type 2 diabetes: the Finnish Diabetes Prevention Study. Diabetes 52, 1872-1876.
Kuipers F, Jong M C, Lin Y, Eck M, Havinga R, Bloks V, Verkade H J, Hofker M H, Moshage H, Berkel T J, Vonk R J, Havekes L M, 1997, Impaired secretion of very low density lipoprotein-triglycerides by apolipoprotein E-deficient mouse hepatocytes. J Clin Invest. 100:2915-2922.
Landolfi, N. F., Cook, R. G., 1986. Activated T-lymphocytes express class I molecules which are hyposialylated compared to other lymphocyte populations. Mol. Immunol. 23, 297-309.
Landolfi, N. F., Leone, J., Womack, J. E., Cook, R. G., 1985. Activation of T lymphocytes results in an increase in H-2-encoded neuraminidase. Immunogenetics 22, 159-167.
Lassar, A. B., Buskin, J. N., Lockshon, D., Davis, R. L., Apone, S., Hauschka, S. D., Weintraub, H., 1989. MyoD is a sequence-specific DNA binding protein requiring a region of myc homology to bind to the muscle creatine kinase enhancer. Cell 58, 823-831.
Liu Y, Berthier-Schaad Y, Fallin M D, Fink N E, Tracy R P, Klag M J, Smith M W, Coresh J. 2006. IL-6 haplotypes, inflammation, and risk for cardiovascular disease in a multiethnic dialysis cohort. J Am Soc Nephrol. 17:863-870
Marmillot P, Rao M N, Liu Q H, Lakshman M R. (1999) Desialylation of human apolipoprotein E decreases its binding to human high-density lipoprotein and its ability to deliver esterified cholesterol to the liver. Metabolism, 48:1184-1192.
Mehrabian, M., Allayee, H., Wong, J., Shi, W., Wang, X. P., Shaposhnik, Z., Funk, C. D., Lusis, A. J., Shih, W., 2002. Identification of 5-lipoxygenase as a major gene contributing to atherosclerosis susceptibility in mice. Circ. Res. 91, 120-126.
Mittal, S. K., Bett, A. J., Prevec, L., Graham, F. L., 1995. Foreign gene expression by human adenovirus type 5-based vectors studied using firefly luciferase and bacterial beta-galactosidase genes as reporters. Virology 210, 226-230.
Miyagi, T., Wada, T., Iwamatsu, A., Hata, K., Yoshikawa, Y., Tokuyama, S., Sawada, M., 1999. Molecular cloning and characterization of a plasma membrane-associated sialidase specific for gangliosides. J. Biol. Chem. 274, 5004-5011.
Monti, E., Bassi, M. T., Bresciani, R., Civini, S., Croci, G. L., Papini, N., Riboni, M., Zanchetti, G., Ballabio, A., Preti, A., Tettamanti, G., Venerando, B., Borsani, G., 2004. Molecular cloning and characterization of NEU4, the fourth member of the human sialidase gene family. Genomics 83, 445-453.
Monti, E., Preti, A., Rossi, E., Ballabio, A., Borsani, G., 1999. Cloning and characterization of NEU2, a human gene homologous to rodent soluble sialidases. Genomics 57, 137-143.
Ng, P., Parks, R. J., Cummings, D. T., Evelegh, C. M., Graham, F. L., 2000. An enhanced system for construction of adenoviral vectors by the two-plasmid rescue method. Hum. Gene Ther. 11, 693-699.
Nishina, P. M., Wang, J., Toyofuku, W., Kuypers, F. A., Ishida, B. Y., Paigen, B., 1993. Atherosclerosis and plasma and liver lipids in nine inbred strains of mice. Lipids 28, 599-605.
O'Brien, J. S., Warner, T. G., 1980. Sialidosis: delineation of subtypes by neuraminidase assay. Clin. Genet. 17, 35-38.
Paigen, B., 1995. Genetics of responsiveness to high-fat and high-cholesterol diets in the mouse. Am. J. Clin. Nutr. 62, 458S462S.
Peltekova, V. D., Wintle, R. F., Rubin, L. A., Amos, C. I., Huang, Q., Gu, X., Newman, B., Van Oene, M., Cescon, D., Greenberg, G., Griffiths, A. M., George-Hyslop, P. H., Siminovitch, K. A., 2004. Functional variants of OCTN cation transporter genes are associated with Crohn disease. Nat. Genet. 36, 471-475.
Peters, J., Swallow, D. M., Andrews, S. J., Evans, L., 1981. A gene (Neu-1) on chromosome 17 of the mouse affects acid alpha-glucosidase and codes for neuraminidase. Genet. Res. 38, 47-55.
Pilatte, Y., Bignon, J., Lambre, C. R., 1993. Sialic acids as important molecules in the regulation of the immune system: pathophysiological implications of sialidases in immunity. Glycobiology 3, 201-218.
Pitman, W. A., Hunt, M. H., McFarland, C., Paigen, B., 1998. Genetic analysis of the difference in diet-induced atherosclerosis between the inbred mouse strains SM/J and NZB/BINJ. Arterioscler. Thromb. Vasc. Biol. 18, 615-620.
Pitman, W. A., Korstanje, R., Churchill, G. A., Nicodeme, E., Albers, J. J., Cheung, M. C., Staton, M. A., Sampson, S. S., Harris, S., Paigen, B., 2002. Quantitative trait locus mapping of genes that regulate HDL cholesterol in SM/J and NZB/B1NJ inbred mice. Physiol Genomics 9, 93-102.
Plump A S, Smith J D, Havek T, Aalto-Setala K, Walsh A. Verstuvft J G. Rubin E M, Breslow J L. 1992. Severe hypercholesterolemia and atherosclerosis in apolipoprotein E-deficient mice created by homologous recombination in ES cells. Cell. 71, 343-353.
Potier, M., Lu Shun, Y. D., Womack, J. E., 1979. Neuraminidase deficiency in the mouse. FEBS Lett. 108, 345-348.
Pshezhetsky, A. V., Richard, C., Michaud, L., Igdoura, S., Wang, S., Elsliger, M. A., Qu, J., Leclerc, D., Gravel, R., Dallaire, L., Potier, M., 1997. Cloning, expression and chromosomal mapping of human lysosomal sialidase and characterization of mutations in sialidosis. Nat. Genet. 15, 316-320.
Purcell-Huynh, D. A., Weinreb, A., Castellani, L. W., Mehrabian, M., Doolittle, M. H., Lusis, A. J., 1995. Genetic factors in lipoprotein metabolism. Analysis of a genetic cross between inbred mouse strains NZB/BINJ and SM/J using a complete linkage map approach. J. Clin. Invest 96, 1845-1858.
Ross, R., 1999. Atherosclerosis—an inflammatory disease. N Engl J. Med. 340, 115-126.
Rottier, R. J., Bonten, E., d'Azzo, A., 1998. A point mutation in the neu-1 locus causes the neuraminidase defect in the SM/J mouse. Hum. Mol. Genet. 7, 313-321.
Sasagasako, N., Shida, N., Yoshimura, T., Kobayashi, T., Goto, I., 1993. [A family with MELAS whose main manifestations are maternally-transmitted deafness and diabetes mellitus]. Rinsho Shinkeigaku 33, 657-659.
Schauer, R., 1982. Chemistry, metabolism, and biological functions of sialic acids. Adv. Carbohydr. Chem. Biochem. 40, 131-234.
Schneider, A., Mijalski, T., Schlange, T., Dai, W., Overbeek, P., Amold, H. H., Brand, T., 1999. The homeobox gene NKX3.2 is a target of left-right signalling and is expressed on opposite sides in chick and mouse embryos. Curr. Biol. 9, 911-914.
Seyrantepe, V., Landry, K., Trudel, S., Hassan, J. A., Morales, C. R., Pshezhetsky, A. V., 2004. Neu4, a novel human lysosomal lumen sialidase confers normal phenotype to sialidosis and galactosialidosis cells. J. Biol. Chem. 279:37021-37029
Schieffer B, Selle T, Hilfiker A, Hilfiker-Kleiner D, Grote K, Tietqe U J, Trautwein C, Luchtefeld M, Schmiftkamp C, Heeneman S. Daemen M J, Drexler H, 2004 Impact of interleukin-6 on plaque development and morphology in experimental atherosclerosis. Circulation. 110:3493-3500.
Smith, J. D., Trogan, E., Ginsberg, M., Grigaux, C., Tian, J., Miyata, M., 1995. Decreased atherosclerosis in mice deficient in both macrophage colony-stimulating factor (op) and apolipoprotein E. Proc. Natl. Acad. Sci. U.S. A 92, 8264-8268.
Song L, Schindler C, 2004. IL-6 and the acute phase response in murine atherosclerosis. Atherosclerosis. 177:43-51.
Sprague, E A, Moser M, Edwards E H, Schwartz C J (1988) Stimulation of receptor-mediated low density lipoprotein endocytosis in neuraminidase-treated cultured bovine aortic endothelial cells. J Cell Physiol. 137(2):251-62.
Steadman, D. J., Giuffrida, D., Gelmann, E. P., 2000. DNA-binding sequence of the human prostate-specific homeodomain protein NKX3.1. Nucleic Acids Res. 28, 2389-2395.
Storer, J. B., 1966. Longevity and gross pathology at death in 22 inbred strains of mice. J. Gerontol. 21, 404-409.
Teriete P, Banerji S, et al. (2004) Structure of the regulatory Hyaluronan binding domain in the inflammatory leukocyte homing receptor CD44. Mol. Cell 13:483-396.
Tertov, V V. Kaplun, I A, Sobenin, Orekhov, AN Low density lipoprotein modification occurring in human plasma. Possible mechanism of in vivo lipoprotein desialylation as a primary step of atherogenic modification. 1998 Atherosclerosis 138, 183-195.
Varki, A., 1997. Sialic acids as ligands in recognition phenomena. FASEB J. 11, 248-255.
Wada, T., Yoshikawa, Y., Tokuyama, S., Kuwabara, M., Akita, H., Miyagi, T., 1999. Cloning, expression, and chromosomal mapping of a human ganglioside sialidase. Biochem. Biophys. Res. Commun. 261, 21-27.
Wang, P., Zhang, J., Bian, H., Wu, P., Kuvelkar, R., Kung, T. T., Crawley, Y., Egan, R. W., Billah, M. M., 2004. Induction of lysosomal and plasma membrane-bound sialidases in human T-cells via T-cell receptor. Biochem. J. 380, 425-433.
Womack, J. E., David, C. S., 1982. Mouse gene for neuraminidase activity (Neu-1) maps to the D end of H-2. Immunogenetics 16, 177-180.
Womack, J. E., Yan, D. L., Potier, M., 1981. Gene for neuraminidase activity on mouse chromosome 17 near h-2: pleiotropic effects on multiple hydrolases. Science 212, 63-65.
Yamamoto, N., Kumashiro, R., 1993. Conversion of vitamin D3 binding protein (group-specific component) to a macrophage activating factor by the stepwise action of beta-galactosidase of B cells and sialidase of T cells. J. Immunol. 151, 2794-2802.
Yamamoto, N., Naraparaju, V. R., 1996. Role of vitamin D3-binding protein in activation of mouse macrophages. J. Immunol. 157, 1744-1749.
Zhang S H, Reddick R L, Piedrahita J A, Maeda N., 1992. Spontaneous hypercholesterolemia and arterial lesions in mice lacking apolipoprotein E. Science. 258:468-471.
Zhang S H, Reddick R L, Burkev B. Maeda N., 1994, Diet-induced atherosclerosis in mice heterozygous and homozygous for apolipoprotein E gene disruption. J Clin Invest. 94:937-945.
Zhou, X. P., Waite, K. A., Pilarski, R., Hampel, H., Femandez, M. J., Bos, C., Dasouki, M., Feldman, G. L., Greenberg, L. A., Ivanovich, J., Matloff, E., Patterson, A., Pierpont, M. E., Russo, D., Nassif, N. T., Eng, C., 2003. Germline PTEN promoter mutations and deletions in Cowden/Bannayan-Riley-Ruvalcaba syndrome result in aberrant PTEN protein and dysregulation of the phosphoinositol-3-kinase/Akt pathway. Am. J. Hum. Genet. 73, 404-411.

1. A method of treating or preventing a condition associated with metabolic syndrome, said method comprising downregulating the expression or activity of a sialidase enzyme in the subject.

2. A method according to claim 1 wherein downregulation is achieved by administering to a subject in need an amount of a sialidase inhibitor effective to treat the condition.

3. A method according to claim 1 wherein the condition is selected from the group consisting of insulin resistance syndrome, diabetes, hyperlipidemia, fatty liver disease, cachexia, obesity, atherosclerosis, and arterioscerlosis.

4. A method according to claim 2 wherein the condition is insulin resistance syndrome.

5. A method according to claim 2 wherein the condition is diabetes.

6. A method according to claim 2 wherein the condition is hyperlipidemia.

7. A method according to claim 2 wherein the condition is fatty liver disease.

8. A method according to claim 2 wherein the condition is cachexia.

9. A method according to claim 2 wherein the condition is obesity.

10. A method according to claim 2 wherein the condition is arterioscerlosis.

11. A method according to claim 2 wherein the condition is atherosclerosis.

12. A method according to claim 1 wherein the sialidase inhibitor is selected from the group consisting of ADDN, Neu5Ac2en, 9-azido-Neu5Ac2en, 9-NANP-Neu5Ac2en and the like.

13. A method according to claim 1 wherein the sialidase inhibitor comprises an antibody that interferes with sialidase activity.

14. A method according to claim 1 wherein the sialidase inhibitor is a nucleic acid.

15. A method according to claim 1 further comprising administering a second agent for the treatment of atherosclerosis or coronary heart disease.

16. Method according to claim 15 wherein the second agent is an acyl CoA:cholesterol acyl transferase inhibitor, an apolipoprotein free acceptor, a statin, a resin or bile acid sequestrant, an inhibitor of cholesterol absorption, niacin, ezetimibe, a liver X receptor agonist, a Ca2+ antagonist or a modulator of peroxisome proliferator-activated receptors.

17. A method according to claim 16 wherein the apolipoprotein free acceptor is cyclodextrin.

18. The method of claim 2, wherein the agent is administered orally.

19. The method of claim 2, wherein the subject is a human.

20. A method according to claim 1 wherein the sialidase inhibitor inhibits the activity or expression of a sialidase peptide or protein encoded by the neu1 gene.

21. The use of a sialidase inhibitor as an agent for the treatment of a metabolic syndrome disorder.

22. The use of a sialidase inhibitor in the manufacture of a medicament for the treatment of a metabolic syndrome disorder.

23. A method for inhibiting and/or inactivating a sialidase enzyme, said method comprising administering a sialidase inhibitor.

24. A method of claim 23 wherein the sialidase inhibitor comprises at least one anti-sialidase antibody or a non-antibody sialidase inhibitor, or a combination of at least one anti-sialidase antibody and at least one non-antibody sialidase inhibitor.

25. The method of claim 24 wherein the non-antibody sialidase inhibitor is a proteinacious inhibitor.

26. A mouse strain that is deficient in neu1 sialidase gene expression.

27. A mouse strain according to claim 26 further comprising a defect in ApoE expression.

28. A pharmaceutical composition for the treatment of atherosclerosis, said composition comprising a sialidase inhibitor, a sialidase inhibitor peptide or mimetic, or a sialidase specific antibody and a pharmaceutically acceptable vehicle.
